(19) United States
(12) Patent Application Publication
(10) Pub. No.: US 2022/0064221 A1
(43) Pub. Date: Mar. 3, 2022

(54) BICYCLIC PEPTIDE LIGANDS SPECIFIC FOR INTEGRIN ALPHA-V-BETA-3

(71) Applicant: BicycleTx Limited, Cambridge (GB)

(72) Inventors: Rachid LANI, Cambridge (GB); Catherine STACE, Cambridge (GB); Daniel TEUFEL, Cambridge (GB); Edward WALKER, Cambridge (GB)

(21) Appl. No.: 17/422,935

(22) PCT Filed: Jan. 15, 2020
(86) PCT No.: PCT/GB2020/050071

§ 371 Date: Jul. 14, 2021

(30) Foreign Application Priority Data
Jan. 15, 2019 (GB) 1900527.1

(51) Int. Cl. C07K 7/60; C07K 7/64; A61K 47/64; C07K 19/00

(52) U.S. Cl. CPC C07K 7/60; C07K 19/00; A61K 47/64; C07K 7/64

(57) ABSTRACT

The present invention relates to polypeptides which are covalently bound to non-aromatic molecular scaffolds such that two or more peptide loops are subtended between attachment points to the scaffold. In particular, the invention describes peptides which are high affinity binders of integrin αvβ3. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by integrin αvβ3.

### FIELD OF THE INVENTION

The present invention relates to polypeptides which are covalently bound to non-aromatic molecular scaffolds such that two or more peptide loops are subtended between attachment points to the scaffold. In particular, the invention describes peptides which are high affinity binders of integrin αvβ3. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by integrin αvβ3.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Δ²; Wu et al. (2007), Science 330, 1066-71), a cyclic peptide with the Arg-Gly-Asp motif binding to integrin αVβ3 (355 Δ²) (Xiong et al. (2002), Science 296 (5565), 151-5) or the cyclic peptide inhibitor upain-1 binding to urokinase-type plasminogen activator (603 Δ²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example 1,1′,1″-(1,3,5-triazinane-1,3,5-triyl)triprop-2-en-1-one (TATA) (Heinis et al (2014) Angewandte Chemie, International Edition 53(6) 1602-1606).

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

According to a first aspect of the invention, there is provided a peptide ligand specific for integrin αvβ3 comprising a polypeptide comprising at least three cysteine residues, separated by at least two loop sequences, and a non-aromatic molecular scaffold which forms covalent bonds with the cysteine residues of the polypeptide such that at least two polypeptide loops are formed on the molecular scaffold.

According to a further aspect of the invention, there is provided a drug conjugate comprising a peptide ligand as defined herein conjugated to one or more effector and/or functional groups.

According to a further aspect of the invention, there is provided a pharmaceutical composition comprising a peptide ligand or a drug conjugate as defined herein in combination with one or more pharmaceutically acceptable excipients.

According to a further aspect of the invention, there is provided a peptide ligand or drug conjugate as defined herein for use in preventing, suppressing or treating a disease or disorder mediated by integrin αvβ3.

### DETAILED DESCRIPTION OF THE INVENTION

In one embodiment, said loop sequences comprise 2, 3, 5, 6, 7 or 9 amino acids.

In a further embodiment, said loop sequences comprise three cysteine residues separated by two loop sequences one of which consists of 2 amino acids and the other of which consists of 7 amino acids.

In a further embodiment, said loop sequences comprise three cysteine residues separated by two loop sequences one of which consists of 3 amino acids and the other of which consists of 6 amino acids.

In a further embodiment, said loop sequences comprise three cysteine residues separated by two loop sequences one of which consists of 3 amino acids and the other of which consists of 7 amino acids.

In a further embodiment, said loop sequences comprise three cysteine residues separated by two loop sequences one of which consists of 3 amino acids and the other of which consists of 9 amino acids.

In a further embodiment, said loop sequences comprise three cysteine residues separated by two loop sequences both of which consist of 5 amino acids.

In a further embodiment, said loop sequences comprise three cysteine residues separated by two loop sequences both of which consist of 6 amino acids.

In one embodiment, the peptide ligand comprises an amino acid sequence selected from:

(SEQ ID NO: 4)
CᵢYDDCᵢᵢRRLDHWQHSCᵢᵢᵢ;
(SEQ ID NO: 23)
Cᵢ-X-H-X-X-R-T/L-D-Cᵢᵢ-X-X-X₁-Cᵢᵢᵢ;
(SEQ ID NO: 24)
Cᵢ-D/E-A/L-S/R-R/H-L/D-D/L-Cᵢᵢ-X-X-X-X-S/H-X-Cᵢᵢᵢ;
(SEQ ID NO: 25)
Cᵢ-P-H-A/L-G-R-Cᵢᵢ-D-G-P-P/L-T/V-Cᵢᵢᵢ;
and
(SEQ ID NO: 26)
Cᵢ-D/H-H/V-X-R-M-D-Cᵢᵢ-P/F-X-X-Cᵢᵢᵢ;

wherein X represents any amino acid and X₁ represents any amino acid or is absent and Cᵢ, Cᵢᵢ and Cᵢᵢᵢ represent first, second and third cysteine residues, respectively or a pharmaceutically acceptable salt thereof.

In a further embodiment, the peptide ligand of Cᵢ-X-H-X-X-R-T/L-D-Cᵢᵢ-X-X-X₁-Cᵢᵢᵢ (SEQ ID NO: 23) comprises an amino acid sequence selected from any one of SEQ ID NOS: 1, 3, 5-6, 8, 10-11, 17 and 20-21:

(SEQ ID NO: 1)
CᵢKHYGRTDCᵢᵢHDTCᵢᵢᵢ;
(SEQ ID NO: 3)
CᵢPHIGRTDCᵢᵢPPCᵢᵢᵢ;
(SEQ ID NO: 5)
CᵢRHSDRLDCᵢᵢLPCᵢᵢᵢ;
(SEQ ID NO: 6)
CᵢPHSLRLDCᵢᵢHDCᵢᵢᵢ;
(SEQ ID NO: 8)
CᵢRHTHRLDCᵢᵢTESCᵢᵢᵢ;
(SEQ ID NO: 10)
CᵢGHVGRLDCᵢᵢHIPCᵢᵢᵢ;
(SEQ ID NO: 11)
CᵢPHVHRLDCᵢᵢHAPCᵢᵢᵢ;
(SEQ ID NO: 17)
CᵢKHSGRTDCᵢᵢHDTCᵢᵢᵢ;
(SEQ ID NO: 20)
CᵢKHAGRTDCᵢᵢPPCᵢᵢᵢ;
and
(SEQ ID NO: 21)
CᵢRHAGRTDCᵢᵢPPCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-D/E-A/L-S/R-R/H-L/D-D/L-Cᵢᵢ-X-X-X-X-S/H-X-Cᵢᵢᵢ (SEQ ID NO: 24) comprises an amino acid sequence selected from any one of SEQ ID NOS: 2, 7 and 18-19:

(SEQ ID NO: 2)
CᵢDASRLDCᵢᵢVPSSSGCᵢᵢᵢ;
(SEQ ID NO: 7)
CᵢELRHDLCᵢᵢRSHDHWCᵢᵢᵢ;
(SEQ ID NO: 18)
CᵢDASRLDCᵢᵢPYSVSLCᵢᵢᵢ;
and
(SEQ ID NO: 19)
CᵢDASRLDCᵢᵢPVVSHLCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-P-H-A/L-G-R-Cᵢᵢ-D-G-P-P/L-T/V-Cᵢᵢᵢ (SEQ ID NO: 25) comprises an amino acid sequence selected from any one of SEQ ID NOS: 9 and 22:

(SEQ ID NO: 9)
CᵢPHAGRCᵢᵢDGPPTCᵢᵢᵢ;
and
(SEQ ID NO: 22)
CᵢPHLGRCᵢᵢDGPLVCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-D/H-H/V-X-R-M-D-Cᵢᵢ-P/F-X-X-Cᵢᵢᵢ (SEQ ID NO: 26) comprises an amino acid sequence selected from any one of SEQ ID NOS: 12-16:

(SEQ ID NO: 12)
CᵢDHRRMDCᵢᵢPEVCᵢᵢᵢ;
(SEQ ID NO: 13)
CᵢDHRRMDCᵢᵢPTLCᵢᵢᵢ;
(SEQ ID NO: 14)
CᵢDHRRMDCᵢᵢPTNCᵢᵢᵢ;
(SEQ ID NO: 15)
CᵢDHTRMDCᵢᵢPHNCᵢᵢᵢ;
and
(SEQ ID NO: 16)
CᵢHVGRMDCᵢᵢFQECᵢᵢᵢ;

wherein Cᵢ, Cᵢᵢ and Cᵢᵢᵢ represent first, second and third cysteine residues, respectively, or a pharmaceutically acceptable salt thereof.

In an alternative embodiment, the peptide ligand of Cᵢ-D/H-H/V-X-R-M-D-Cᵢᵢ-P/F-X-X-Cᵢᵢᵢ (SEQ ID NO: 26) comprises an amino acid sequence selected from;

(SEQ ID NO: 27)
CᵢDHRRMDCᵢᵢPTACᵢᵢᵢ;

wherein Cᵢ, Cᵢᵢ and Cᵢᵢᵢ represent first, second and third cysteine residues, respectively, or a pharmaceutically acceptable salt thereof.

In an alternative embodiment, the peptide ligand is selected from:

(SEQ ID NO: 28)
CᵢHATRNMDCᵢᵢYTCᵢᵢᵢ;
(SEQ ID NO: 29)
CᵢPHLERLDCᵢᵢHDSCᵢᵢᵢ;
(SEQ ID NO: 30)
CᵢHKGRGDHCᵢᵢLTCᵢᵢᵢ;
(SEQ ID NO: 31)
CᵢSPLRMDCᵢᵢHTVSDTCᵢᵢᵢ;
(SEQ ID NO: 32)
CᵢHSFRTDCᵢᵢHNHCᵢᵢᵢ;
(SEQ ID NO: 33)
CᵢPESHYLCᵢᵢRLDHHHCᵢᵢᵢ;
(SEQ ID NO: 34)
CᵢHAYRTDCᵢᵢHDYCᵢᵢᵢ;
(SEQ ID NO: 35)
CᵢNPRSDAPCᵢᵢEPCᵢᵢᵢ;
(SEQ ID NO: 36)
CᵢRTDDYRDCᵢᵢDICᵢᵢᵢ;
(SEQ ID NO: 37)
CᵢPHI[dA]RTDCᵢᵢPPCᵢᵢᵢ;
(SEQ ID NO: 38)
CᵢPHIG[Agb]TDCᵢᵢPPCᵢᵢᵢ;
(SEQ ID NO: 39)
CᵢDAS[HArg]LDCᵢᵢVPSSS[dA]Cᵢᵢᵢ;
(SEQ ID NO: 40)
CᵢP[HArg]I[dA]RTDCᵢᵢPPCᵢᵢᵢ;
(SEQ ID NO: 41)
CᵢP[HArg]IG[HArg]TDCᵢᵢPPCᵢᵢᵢ;
(SEQ ID NO: 42)
CᵢPHIG[HArg]TDCᵢᵢPPCᵢᵢᵢ;
(SEQ ID NO: 43)
CᵢP[HArg]IGRTDCᵢᵢPPCᵢᵢᵢ;
(SEQ ID NO: 44)
CᵢGHV[dA]RLDCᵢᵢHIPCᵢᵢᵢ;
(SEQ ID NO: 45)
CᵢGHVG[HArg]LDCᵢᵢHIPCᵢᵢᵢ;
(SEQ ID NO: 46)
Cᵢ[dA]HVGRLDCᵢᵢHIPCᵢᵢᵢ;
(SEQ ID NO: 47)
CᵢG[HArg]V[dA]RLDCᵢᵢ[HArg]IPCᵢᵢᵢ;
(SEQ ID NO: 48)
CᵢGHVGRLDCᵢᵢ[HArg]IPCᵢᵢᵢ;
(SEQ ID NO: 49)
Cᵢ[dA]HV[dA]RLDCᵢᵢ[HArg]IPCᵢᵢᵢ;
(SEQ ID NO: 50)
CᵢGHV[dA]RLDCᵢᵢ[HArg]IPCᵢᵢᵢ;
(SEQ ID NO: 51)
CᵢGHVG[HArg]LDCᵢᵢ[HArg]IPCᵢᵢᵢ;
(SEQ ID NO: 52)
Cᵢ[dA]HVG[HArg]LDCᵢᵢHIPCᵢᵢᵢ;
(SEQ ID NO: 53)
CᵢDAS[HArg]LDCᵢᵢVPSSSGCᵢᵢᵢ;
and
(SEQ ID NO: 54)
CᵢHTRAHDCᵢᵢYWESIVCᵢᵢᵢ;

wherein Agb represents 2-amino-4-guanidinobutyric acid, HArg represents homoarginine, Cᵢ, Cᵢᵢ and Cᵢᵢᵢ represent first, second and third cysteine residues, respectively, or a pharmaceutically acceptable salt thereof.

In a further embodiment, the peptide ligand comprises an amino acid sequence selected from:
A-(SEQ ID NO: 1)-A (herein referred to as BCY2519);
A-(SEQ ID NO: 2)-A (herein referred to as BCY2521);
A-(SEQ ID NO: 3)-A (herein referred to as BCY2524);
A-(SEQ ID NO: 4) (herein referred to as BCY2527);
A-(SEQ ID NO: 5) (herein referred to as BCY2528);
A-(SEQ ID NO: 6)-A (herein referred to as BCY2533);
A-(SEQ ID NO: 7)-A (herein referred to as BCY2534);
A-(SEQ ID NO: 8)-A (herein referred to as BCY2540);
A-(SEQ ID NO: 9)-A (herein referred to as BCY2541);
A-(SEQ ID NO: 10)-A (herein referred to as BCY2550);
A-(SEQ ID NO: 11)-A (herein referred to as BCY2552);
A-(SEQ ID NO: 12)-A (herein referred to as BCY2544);
A-(SEQ ID NO: 13)-A (herein referred to as BCY2545);
A-(SEQ ID NO: 14)-A (herein referred to as BCY2546);
A-(SEQ ID NO: 15)-A (herein referred to as BCY2547);
A-(SEQ ID NO: 16)-A (herein referred to as BCY2548);
A-(SEQ ID NO: 17)-A (herein referred to as BCY2520);
A-(SEQ ID NO: 18)-A (herein referred to as BCY2522);
A-(SEQ ID NO: 19)-A (herein referred to as BCY2523);
A-(SEQ ID NO: 20)-A (herein referred to as BCY2525);
A-(SEQ ID NO: 21)-A (herein referred to as BCY2526); and
A-(SEQ ID NO: 22)-A (herein referred to as BCY2542).

In an alternative embodiment, the peptide ligand comprises an amino acid sequence selected from:
A-(SEQ ID NO: 27)-A (herein referred to as BCY2543).

In one embodiment, the molecular scaffold is selected from 1,1′,1″-(1,3,5-triazinane-1,3,5-triyl)triprop-2-en-1-one (TATA) and the peptide ligand comprises an amino acid sequence selected from:
A-(SEQ ID NO: 1)-A (herein referred to as BCY2519);
A-(SEQ ID NO: 2)-A (herein referred to as BCY2521);
A-(SEQ ID NO: 3)-A (herein referred to as BCY2524);
A-(SEQ ID NO: 4) (herein referred to as BCY2527);
A-(SEQ ID NO: 5) (herein referred to as BCY2528);
A-(SEQ ID NO: 6)-A (herein referred to as BCY2533);
A-(SEQ ID NO: 7)-A (herein referred to as BCY2534);
A-(SEQ ID NO: 8)-A (herein referred to as BCY2540);
A-(SEQ ID NO: 9)-A (herein referred to as BCY2541);
A-(SEQ ID NO: 10)-A (herein referred to as BCY2550);
A-(SEQ ID NO: 11)-A (herein referred to as BCY2552);
A-(SEQ ID NO: 12)-A (herein referred to as BCY2544);
A-(SEQ ID NO: 13)-A (herein referred to as BCY2545);
A-(SEQ ID NO: 14)-A (herein referred to as BCY2546);
A-(SEQ ID NO: 15)-A (herein referred to as BCY2547);
A-(SEQ ID NO: 16)-A (herein referred to as BCY2548);
A-(SEQ ID NO: 17)-A (herein referred to as BCY2520);
A-(SEQ ID NO: 18)-A (herein referred to as BCY2522);
A-(SEQ ID NO: 19)-A (herein referred to as BCY2523);
A-(SEQ ID NO: 20)-A (herein referred to as BCY2525);
A-(SEQ ID NO: 21)-A (herein referred to as BCY2526); and
A-(SEQ ID NO: 22)-A (herein referred to as BCY2542).

In an alternative embodiment, the molecular scaffold is selected from 1,1′,1″-(1,3,5-triazinane-1,3,5-triyl)triprop-2-en-1-one (TATA) and the peptide ligand comprises an amino acid sequence selected from:
A-(SEQ ID NO: 27)-A (herein referred to as BCY2543).

In a further embodiment, the molecular scaffold is selected from 1,1′,1″-(1,3,5-triazinane-1,3,5-triyl)triprop-2-en-1-one (TATA) and the peptide ligand comprises an amino acid sequence selected from:
A-(SEQ ID NO: 4) (herein referred to as 43-26-00-N002);
A-(SEQ ID NO: 5) (herein referred to as 43-27-00-N002);
A-(SEQ ID NO: 6)-A (herein referred to as 43-32-00-N002);
A-(SEQ ID NO: 7)-A (herein referred to as 43-33-00-N002);
A-(SEQ ID NO: 8)-A (herein referred to as 43-39-00-N002);
A-(SEQ ID NO: 9)-A (herein referred to as 43-40-00-N002);
A-(SEQ ID NO: 12)-A (herein referred to as 43-41-01-N001);
A-(SEQ ID NO: 13)-A (herein referred to as 43-41-02-N001);
A-(SEQ ID NO: 14)-A (herein referred to as 43-41-03-N001); and
A-(SEQ ID NO: 19)-A (herein referred to as 43-24-02-N001).

The scaffold/peptide ligands of this embodiment demonstrated superior integrin αvβ3 competition binding as shown herein in Table 1.

In one embodiment, the peptide ligand additionally comprises a fluorescent moiety such as fluorescein (Fl) or Cyanine5 (Cy5).

In a further embodiment, the peptide ligand additionally comprises a fluorescent moiety such as fluorescein (Fl) or Cyanine5 (Cy5) and is selected from:
A-(SEQ ID NO: 1)-A-Sar₆-K-Fl (herein referred to as BCY2594);
A-(SEQ ID NO: 2)-A-Sar₆-K-Fl (herein referred to as BCY2595);
Ac-(SEQ ID NO: 2)-A-Sar₆-K-Cy5 (herein referred to as BCY8589);
A-(SEQ ID NO: 3)-A-Sar₆-K-Fl (herein referred to as BCY2596);
Ac-(SEQ ID NO: 3)-A-Sar₆-K-Fl (herein referred to as BCY7508);
(SEQ ID NO: 4)-A-Sar₆-K-Fl (herein referred to as BCY2597);
A-(SEQ ID NO: 5)-A-Sar₆-K-Fl (herein referred to as BCY2598);
A-(SEQ ID NO: 6)-A-Sar₆-K-Fl (herein referred to as BCY2603);
A-(SEQ ID NO: 7)-A-Sar₆-K-Fl (herein referred to as BCY2604);
A-(SEQ ID NO: 8)-A-Sar₆-K-Fl (herein referred to as BCY2610);
Ac-(SEQ ID NO: 10)-A-Sar₆-K-Fl (herein referred to as BCY7509);
A-(SEQ ID NO: 28)-A-Sar₆-K-Fl (herein referred to as BCY2599);
A-(SEQ ID NO: 29)-A-Sar₆-K-Fl (herein referred to as BCY2600);
A-(SEQ ID NO: 30)-A-Sar₆-K-Fl (herein referred to as BCY2601);
A-(SEQ ID NO: 31)-A-Sar₆-K-Fl (herein referred to as BCY2602);
A-(SEQ ID NO: 32)-A-Sar₆-K-Fl (herein referred to as BCY2605);
A-(SEQ ID NO: 33)-A-Sar₆-K-Fl (herein referred to as BCY2606);
A-(SEQ ID NO: 34)-A-Sar₆-K-Fl (herein referred to as BCY2607);
A-(SEQ ID NO: 35)-A-Sar₆-K-Fl (herein referred to as BCY2608);
A-(SEQ ID NO: 36)-A-Sar₆-K-Fl (herein referred to as BCY2609);
Ac-(SEQ ID NO: 37)-A-Sar₆-K-Fl (herein referred to as BCY7503);
Ac-(SEQ ID NO: 37)-A-Sar₆-K-Cy5 (herein referred to as BCY8593);
Ac-(SEQ ID NO: 38)-A-Sar₆-K-Fl (herein referred to as BCY7502);
Ac-(SEQ ID NO: 39)-A-Sar₆-K-Fl (herein referred to as BCY7501);
Ac-(SEQ ID NO: 40)-A-Sar₆-K-Fl (herein referred to as BCY7504);
Ac-(SEQ ID NO: 41)-A-Sar₆-K-Fl (herein referred to as BCY7505);
Ac-(SEQ ID NO: 42)-A-Sar₆-K-Fl (herein referred to as BCY7506);
Ac-(SEQ ID NO: 43)-A-Sar₆-K-Fl (herein referred to as BCY7507);
Ac-(SEQ ID NO: 44)-A-Sar₆-K-Fl (herein referred to as BCY7510);
Ac-(SEQ ID NO: 44)-A-Sar₆-K-Cy5 (herein referred to as BCY8592);
Ac-(SEQ ID NO: 45)-A-Sar₆-K-Fl (herein referred to as BCY7511);
Ac-(SEQ ID NO: 46)-A-Sar₆-K-Fl (herein referred to as BCY7512);
Ac-(SEQ ID NO: 47)-A-Sar₆-K-Fl (herein referred to as BCY7513);
Ac-(SEQ ID NO: 48)-A-Sar₆-K-Fl (herein referred to as BCY7514);
Ac-(SEQ ID NO: 49)-A-Sar₆-K-Fl (herein referred to as BCY7515);
Ac-(SEQ ID NO: 50)-A-Sar₆-K-Fl (herein referred to as BCY7516);
Ac-(SEQ ID NO: 51)-A-Sar₆-K-Fl (herein referred to as BCY7517);
Ac-(SEQ ID NO: 52)-A-Sar₆-K-Fl (herein referred to as BCY7518);
Ac-(SEQ ID NO: 53)-A-Sar₆-K-Fl (herein referred to as BCY7764); and
Ac-(SEQ ID NO: 54)-A-Sar₆-K-Cy5 (herein referred to as BCY8588).

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

### Nomenclature

### Numbering

When referring to amino acid residue positions within the peptides of the invention, cysteine residues (Cᵢ, Cᵢᵢ and Cᵢᵢᵢ) are omitted from the numbering as they are invariant, therefore, the numbering of amino acid residues within the peptides of the invention is referred to as below:

(SEQ ID NO: 1)
-Cᵢ-K₁-H₂-Y₃-G₄-R₅-T₆-D₇-Cᵢᵢ-H₈-D₉-T₁₀-Cᵢᵢᵢ-.

For the purpose of this description, all bicyclic peptides are assumed to be cyclised with 1,1′,1″-(1,3,5-triazinane-1,3,5-triyl)triprop-2-en-1-one (TATA) and yielding a tri-substituted structure. Cyclisation with TATA occurs on Cᵢ, Cᵢᵢ, and Cᵢᵢᵢ.

### Molecular Format

N- or C-terminal extensions to the bicycle core sequence are added to the left or right side of the sequence, separated by a hyphen. For example, an N-terminal βAla-Sar10-Ala tail would be denoted as:

(SEQ ID NO: X)
βAla-Sar10-A-.

### Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

### Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;

Desirable solubility profile. This is a function of the proportion of charged and hydrophilic versus hydrophobic residues and intra/inter-molecular H-bonding, which is important for formulation and absorption purposes;

An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and

Selectivity. Certain peptide ligands of the invention demonstrate good selectivity over other integrins. Certain peptide ligands of the invention demonstrate good selectivity over other integrins, such as αvβ5. In particular, bicyclic peptides BCY2541, BCY2543, BCY2550 and BCY2552 demonstrated selectivity for αvβ3 over αvβ5 in the competition binding assay as shown in Table 2 herein.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand;

replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated.

This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Cα-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:

Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.
   (for reviews see Gentilucci et al, Curr. Pharmaceutical Design, (2010), 16, 3185-203, and Nestor et al, Curr. Medicinal Chem (2009), 16, 4399-418).

### Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the integrin αvβ3 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Non-Aromatic Molecular Scaffold

References herein to the term “non-aromatic molecular scaffold” refer to any molecular scaffold as defined herein which does not contain an aromatic (i.e. unsaturated) carbocyclic or heterocyclic ring system.

Suitable examples of non-aromatic molecular scaffolds are described in Heinis et al (2014) Angewandte Chemie, International Edition 53(6) 1602-1606.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

An example of an αβ unsaturated carbonyl containing compound is 1,1′,1″-(1,3,5-triazinane-1,3,5-triyl)triprop-2-en-1-one (TATA) (Angewandte Chemie, International Edition (2014), 53(6), 1602-1606).

### Effector and Functional Groups

According to a further aspect of the invention, there is provided a drug conjugate comprising a peptide ligand as defined herein conjugated to one or more effector and/or functional groups.

Effector and/or functional groups can be attached, for example, to the N and/or C termini of the polypeptide, to an amino acid within the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains.

An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and Harrison, Biochemical Society Transactions (2007) Volume 35, part 4, p 821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p 10444), the 18 amino acid ‘model amphipathic peptide’ (Oehlke et al (1998) Biochim Biophys Acts Volume 1414 p 127) and arginine rich regions of the HIV TAT protein. Non peptidic approaches include the use of small molecule mimics or SMOCs that can be easily attached to biomolecules (Okuyama et al (2007) Nature Methods Volume 4 p 153). Other chemical strategies to add guanidinium groups to molecules also enhance cell penetration (Elson-Scwab et al (2007) J Biol Chem Volume 282 p 13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half life in the range 12 to 60 hours. In a further embodiment, it will have a tβ half-life of a day or more. In a further embodiment still, it will be in the range 12 to 26 hours.

In one particular embodiment of the invention, the functional group is selected from a metal chelator, which is suitable for complexing metal radioisotopes of medicinal relevance.

Possible effector groups also include enzymes, for instance such as carboxypeptidase G2 for use in enzyme/prodrug therapy, where the peptide ligand replaces antibodies in ADEPT.

In one particular embodiment of the invention, the functional group is selected from a drug, such as a cytotoxic agent for cancer therapy. Suitable examples include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin, calicheamycins, and others.

In one further particular embodiment of the invention, the cytotoxic agent is selected from maytansinoids (such as DM1) or monomethyl auristatins (such as MMAE).

DM1 is a cytotoxic agent which is a thiol-containing derivative of maytansine and has the following structure:

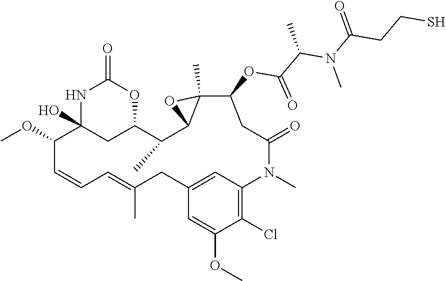 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@@H](OC)/C=C\C=C(/C)C2]

Monomethyl auristatin E (MMAE) is a synthetic antineoplastic agent and has the following structure:

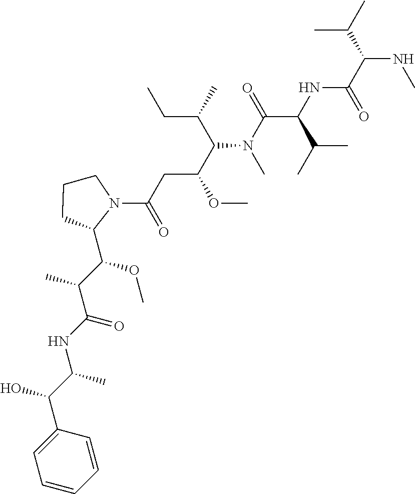

In one embodiment, the cytotoxic agent is linked to the bicyclic peptide by a cleavable bond, such as a disulphide bond or a protease sensitive bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

In one embodiment, the cytotoxic agent and linker is selected from any combinations of those described in WO 2016/067035 (the cytotoxic agents and linkers thereof are herein incorporated by reference).

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a peptide ligand or a drug conjugate as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cyclosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

The bicyclic peptides of the invention have specific utility as integrin αvβ3 binding agents.

Integrins are heterodimeric matrix receptors that anchor cells to substrates and transmit externally derived signals across the plasma membrane. Integrin αvβ3 is involved in the osteoclast-mediated bone resorption, both in vivo and in vitro. This heterodimer molecule recognizes the amino acid motif Arg-Gly-Asp (RGD) contained in bone matrix proteins such as osteopontin and bone sialoprotein. Integrin αvβ3 is expressed in an osteoclast and its expression is modulated by resorptive steroids and cytokines. Based on blocking experiments, αvβ3 integrin has been identified as a major functional adhesion receptor on osteoclasts. Inhibitors of integrin αvβ3 reduce the capacity of osteoclasts to bind to and resorb bone. Integrin αvβ3 plays a major role in the function of osteoclasts and inhibitors of this integrin are being considered for treating or preventing osteoporosis, osteolytic metastases, and malignancy-induced hypercalcemia.

There are many bone diseases that are related to osteolysis that is mediated by osteoclasts. Osteoporosis is the most common one that is induced when resorption and formation of bone are not coordinated and bone breakdown overrides bone building. Osteoporosis is also caused by other conditions, such as hormonal imbalance, diseases, or medications (e.g., corticosteroids or anti-epileptic agents). Bone is one of the most common sites of metastasis by human breast, prostate, lung and thyroid cancers, as well as other cancers. Osteoporosis may also result from post-menopausal estrogen deficiency. Secondary osteoporosis may be associated with rheumatoid arthritis. Bone metastasis shows a very unique step of osteoclastic bone resorption that is not seen in metastasis of other organs. It is widely accepted that osteolysis that is associated with cancer is essentially mediated by osteoclasts, which seem to be activated and may be indirectly activated through osteoblasts or directly by tumor products. In addition, hypercalcemia (increased blood-calcium concentration) is an important complication of osteolytic bone diseases. It occurs relatively frequently in patients with extensive bone destruction, and is particularly common in breast, lung, renal, ovarian and pancreatic carcinomas and in myeloma.

Disintegrins are a family of low-molecular-weight RGD-containing peptides that bind specifically to integrins αIIbβ3, α5β1 and αvβ3 expressed on platelets and other cells including vascular endothelial cells and some tumor cells. In addition to their potent antiplatelet activity, studies of disintegrins have revealed new uses in the diagnosis of cardiovascular diseases and the design of therapeutic agents in arterial thrombosis, osteoporosis and angiogenesis-related tumor growth and metastasis. Rhodostomin (Rho), a disintegrin derived from the venom of Colloselasma rhodostoma, has been found to inhibit platelet aggregation in vivo and in vitro through the blockade of platelet glycoprotein αIIbβ3.

The role of αvβ3 integrin in bone diseases has been well documented (Ross et al (2006) Journal of Clinical Investigation 116(5); Rodan et al (1997) Journal of Endocrinology 154, S47-S56; Teitelbaum (2005) Journal of Clinical Endocrinology and Metabolism 90(4), 2466-2468; Teitelbaum (2000) Journal of Bone and Mineral Metabolism 18, 344-349; Nakamura et al (2007) Journal of Bone and Mineral Metabolism 25, 337-344; Duong et al (1999) Journal of Bone and Mineral Metabolism 17, 1-6; and Teti et al (2002) Calcified Tissue International 71, 293-299). In addition to bone diseases, αvβ3 integrin plays an important role in angiogenesis and tumor growth in conditions not related to bone diseases.

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand or a drug conjugate as defined herein, for use in preventing, suppressing or treating a disease or disorder mediated by integrin αvβ3.

According to a further aspect of the invention, there is provided a method of preventing, suppressing or treating a disease or disorder mediated by integrin αvβ3, which comprises administering to a patient in need thereof an effector group and drug conjugate of the peptide ligand as defined herein.

In one embodiment, the integrin αvβ3 is mammalian integrin αvβ3. In a further embodiment, the mammalian integrin αvβ3 is human integrin αvβ3.

In one embodiment, the disease or disorder mediated by integrin αvβ3 is selected from bone disease (such as osteoporosis), cancer, and diseases involving angiogenesis.

In a further embodiment, the disease or disorder mediated by integrin αvβ3 is selected from cancer.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In a further embodiment, the cancer is selected from cancer of the breast, lung, kidney, ovary and pancreas and myeloma.

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

### Materials and Methods

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology. Peptides were purified using HPLC and following isolation they were modified with 1,3,5-Triacryloylhexahydro-1,3,5-triazine (TATA, Sigma). For this, linear peptide was diluted with 50:50 MeCN:H₂O up to ˜35 mL, ˜500 μL of 100 mM TATA in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Once completed, 1 ml of 1M L-cysteine hydrochloride monohydrate (Sigma) in H₂O was added to the reaction for ˜60 min at RT to quench any excess TATA.

Following lyophilisation, the modified peptide was purified as above, while replacing the Luna C8 with a Gemini C18 column (Phenomenex), and changing the acid to 0.1% trifluoroacetic acid. Pure fractions containing the correct TATA-modified material were pooled, lyophilised and kept at −20° C. for storage.

All amino acids, unless noted otherwise, were used in the L-configurations.

In some cases peptides are converted to activated disulfides prior to coupling with the free thiol group of a toxin using the following method; a solution of 4-methyl(succinimidyl 4-(2-pyridylthio)pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

### Biological Data

Integrin αvβ3 Competition Binding Assay

Affinity of the peptides of the invention for integrin αvβ3 (Ki) was determined using a competition fluorescence polarisation assay analogous to that described in Wang et al (2005) Bioconjug Chem 16(3), 729-34 using 5 nM peptide with the sequence: FITC-Ahx-GRGDSP (FITC-Ahx-(SEQ ID NO: 55) herein after referred to as BCY10185) as the ligand. FITC is 3′,6′-dihydroxy-3-oxospiro[isobenzofuran-1(3H),9′-[9H]xanthene and Ahx is aminohexanoic acid.

The peptide ligands of the invention were tested in the above mentioned integrin αvβ3 competition binding assay and the results are shown in Table 1:

TABLE 1
Biological Assay Data for Peptide Ligands of the Invention

| | Molecular | | | |
|---|---|---|---|---|
| Peptide | Scaffold | Ki (nM) | Mean | n |
| BCY2519 | TATA | 763 | — | 1 |
| BCY2521 | TATA | 121.15 ± 166.3 | 86.5 | 2 |
| BCY2524 | TATA | 126.3 ± 93.49 | 116.9 | 2 |
| BCY2527 | TATA | 33.5 | — | 1 |
| BCY2528 | TATA | 10.7 | — | 1 |
| BCY2533 | TATA | 29.5 | — | 1 |
| BCY2534 | TATA | 81.9 | — | 1 |
| BCY2540 | TATA | 19.1 | — | 1 |
| BCY2541 | TATA | 1090.33 ± 97.21 | 1088.1 | 3 |
| BCY2550 | TATA | 864 ± 197.96 | 858.1 | 2 |
| BCY2552 | TATA | 727 ± 101.92 | 725.1 | 2 |
| BCY2544 | TATA | 38 | — | 1 |
| BCY2545 | TATA | 42 | — | 1 |
| BCY2546 | TATA | 52 | — | 1 |
| BCY2547 | TATA | 107 | — | 1 |
| BCY2548 | TATA | 563 | — | 1 |
| BCY2520 | TATA | 613 | — | 1 |
| BCY2522 | TATA | 204 | — | 1 |
| BCY2523 | TATA | 93 | — | 1 |
| BCY2525 | TATA | 123 | — | 1 |
| BCY2526 | TATA | 153 | — | 1 |
| BCY2542 | TATA | 682 | — | 1 |

Further bicyclic peptides were tested in the above mentioned integrin αvβ3 competition binding assay using the following tracers as competing ligands:
BCY3844—(Galacto-RGD)2-AF488 (prepared according to the method of Colombo et al (2010) Molecules 15(1), 178-197);
BCY2572—an αvβ3 binding bicyclic peptide complexed with TBMB: ACLDHMECRGDMDCA-Sar₆-K-Fl ((SEQ ID NO: 56)-Sar₆-K-Fl);
BCY2576—an αvβ3 binding bicyclic peptide complexed with TBMB: ACILRPNCDLDGRCA-Sar₆-K-Fl ((SEQ ID NO: 57)-Sar₆-K-Fl); and
BCY10185—FITC-Ahx-(SEQ ID NO: 55).

Certain peptide ligands of the invention were tested in the above mentioned integrin αvβ3 and αvβ5 competition binding assays and the results are shown in Table 2:

TABLE 2
Competition Binding Data for Peptide Ligands of the Invention

| | Molecular | | | | | |
|---|---|---|---|---|---|---|
| Peptide | Scaffold | Tracer | Target | Ki (nM) | Mean | n |
| BCY2521 | TATA | BCY2576 | αvβ3 | 103 | — | 1 |
| BCY2524 | TATA | BCY2576 | αvβ3 | 454 | — | 1 |
| BCY2527 | TATA | BCY2576 | αvβ3 | 211 | — | 1 |
| BCY2528 | TATA | BCY2576 | αvβ3 | 91.6 | — | 1 |
| BCY2533 | TATA | BCY2576 | αvβ3 | 194 | — | 1 |
| BCY2534 | TATA | BCY2576 | αvβ3 | 417 | — | 1 |
| BCY2540 | TATA | BCY2576 | αvβ3 | 124 | — | 1 |
| BCY2541 | TATA | BCY3844 | αvβ3 | 111 | — | 1 |
| | | BCY3844 | αvβ5 | >4800 | — | 2 |
| BCY2543 | TATA | BCY3844 | αvβ3 | 3.3 | — | 1 |
| | | BCY3844 | αvβ5 | >4800 | — | 2 |
| | | BCY10185 | αvβ3 | 27.23 ± 17.92 | 23.3 | 4 |
| | | BCY2576 | αvβ3 | 94.9 | — | 1 |
| BCY2550 | TATA | BCY3844 | αvβ3 | 47.9 | — | 1 |
| | | BCY3844 | αvβ5 | >4800 | — | 2 |
| BCY2552 | TATA | BCY3844 | αvβ3 | 26.2 | — | 1 |
| | | BCY3844 | αvβ5 | >4800 | — | 2 |

Integrin αvβ3 and αvβ5 Direct Binding Assay

Affinity of selected fluorescently modified peptides of the invention for integrin αvβ3 (Kd) (and αvβ5 to test for selectivity) was determined using a human direct binding assay analogous to that described in Schottelius et al. (2009) Acc. Chem. Res. 42, 969-980. The results of the direct binding assay are shown in Table 3:

TABLE 3
Direct Binding Data for Peptide Ligands of the Invention

| Peptide | Assay | Target | Kd (nM) | n |
|---|---|---|---|---|
| BCY2594 | Human FP Direct binding | Integrin aVb3 | 5.54 | 1 |
| BCY2595 | Human FP Direct binding | Integrin aVb3 | 5.59 | 1 |
| BCY2596 | Human FP Direct binding | Integrin aVb3 | 6.7 | 1 |
| BCY2597 | Human FP Direct binding | Integrin aVb3 | 5.42 | 1 |
| BCY2598 | Human FP Direct binding | Integrin aVb3 | 5.19 | 1 |
| BCY2599 | Human FP Direct binding | Integrin aVb3 | 7.24 | 1 |
| BCY2600 | Human FP Direct binding | Integrin aVb3 | 7.08 | 1 |
| BCY2601 | Human FP Direct binding | Integrin aVb3 | 5.77 | 1 |
| BCY2602 | Human FP Direct binding | Integrin aVb3 | 8.01 | 1 |
| BCY2603 | Human FP Direct binding | Integrin aVb3 | 10.4 | 1 |
| BCY2604 | Human FP Direct binding | Integrin aVb3 | 5.12 | 1 |
| BCY2605 | Human FP Direct binding | Integrin aVb3 | 3.57 | 1 |
| BCY2606 | Human FP Direct binding | Integrin aVb3 | 5.74 | 1 |
| BCY2607 | Human FP Direct binding | Integrin aVb3 | 5.74 | 1 |
| BCY2608 | Human FP Direct binding | Integrin aVb3 | 11.4 | 1 |
| BCY2609 | Human FP Direct binding | Integrin aVb3 | 4.96 | 1 |
| BCY2610 | Human FP Direct binding | Integrin aVb3 | 7.89 | 1 |
| BCY7503 | Human FP Direct binding | Integrin aVb3 | 0.036 | 1 |
| BCY7502 | Human FP Direct binding | Integrin aVb3 | 8.064 | 1 |
| BCY7501 | Human FP Direct binding | Integrin aVb3 | 11.5 | 1 |
| BCY7504 | Human FP Direct binding | Integrin aVb3 | 1.493 | 1 |
| BCY7505 | Human FP Direct binding | Integrin aVb3 | 0.016 | 1 |
| BCY7506 | Human FP Direct binding | Integrin aVb3 | 7.491 | 1 |
| BCY7507 | Human FP Direct binding | Integrin aVb3 | 61.89 | 1 |
| BCY7508 | Human FP Direct binding | Integrin aVb3 | 0.05 | 1 |
| BCY7509 | Human FP Direct binding | Integrin aVb3 | 0.017 | 1 |
| BCY7510 | Human FP Direct binding | Integrin aVb3 | 0.014 | 1 |
| BCY7511 | Human FP Direct binding | Integrin aVb3 | 0.074 | 1 |
| BCY7512 | Human FP Direct binding | Integrin aVb3 | 0.017 | 1 |
| BCY7513 | Human FP Direct binding | Integrin aVb3 | 0 | 1 |
| BCY7514 | Human FP Direct binding | Integrin aVb3 | 0.159 | 1 |
| BCY7515 | Human FP Direct binding | Integrin aVb3 | 1.666 | 1 |
| BCY7516 | Human FP Direct binding | Integrin aVb3 | 0.028 | 1 |
| BCY7517 | Human FP Direct binding | Integrin aVb3 | 26.28 | 1 |
| BCY7518 | Human FP Direct binding | Integrin aVb3 | 0.089 | 1 |
| BCY7764 | Human FP Direct binding | Integrin aVb3 | 15 | 1 |
| BCY7766* | Human FP Direct binding | Integrin aVb3 | 1.145 | 6 |
| BCY8588 | Human FP Direct binding | Integrin aVb3 | 0.0595 | 2 |
| BCY8589 | Human FP Direct binding | Integrin aVb3 | 0.042 | 2 |
| BCY8592 | Human FP Direct binding | Integrin aVb3 | 0.0285 | 2 |
| BCY8593 | Human FP Direct binding | Integrin aVb3 | 0.0295 | 2 |

*BCY7766 is a linear peptide having sequence FITC-Ahx-GRGSSP (FITC-Ahx-(SEQ ID NO: 58)).

1. A peptide ligand specific for integrin αvβ3 comprising a polypeptide comprising at least three cysteine residues, separated by at least two loop sequences, and a non-aromatic molecular scaffold which forms covalent bonds with the cysteine residues of the polypeptide such that at least two polypeptide loops are formed on the molecular scaffold.

2. The peptide ligand as defined in claim 1, wherein said loop sequences comprise 2, 3, 5, 6, 7 or 9 amino acids.

3. The peptide ligand as defined in claim 1 or claim 2, wherein said loop sequences comprise three cysteine residues separated by two loop sequences one of which consists of 2 amino acids and the other of which consists of 7 amino acids.

4. The peptide ligand as defined in claim 1 or claim 2, wherein said loop sequences comprise three cysteine residues separated by two loop sequences one of which consists of 3 amino acids and the other of which consists of 6 amino acids.

5. The peptide ligand as defined in claim 1 or claim 2, wherein said loop sequences comprise three cysteine residues separated by two loop sequences one of which consists of 3 amino acids and the other of which consists of 7 amino acids.

6. The peptide ligand as defined in claim 1 or claim 2, wherein said loop sequences comprise three cysteine residues separated by two loop sequences one of which consists of 3 amino acids and the other of which consists of 9 amino acids.

7. The peptide ligand as defined in claim 1 or claim 2, wherein said loop sequences comprise three cysteine residues separated by two loop sequences both of which consist of 5 amino acids.

8. The peptide ligand as defined in claim 1 or claim 2, wherein said loop sequences comprise three cysteine residues separated by two loop sequences both of which consist of 6 amino acids.

9. The peptide ligand as defined in claim 1 or claim 2, which comprises an amino acid sequence selected from:
(SEQ ID NO: 4)
CᵢYDDCᵢᵢRRLDHWQHSCᵢᵢᵢ;
(SEQ ID NO: 23)
Cᵢ-X-H-X-X-R-T/L-D-Cᵢᵢ-X-X-X₁-Cᵢᵢᵢ;
(SEQ ID NO: 24)
Cᵢ-D/E-A/L-S/R-R/H-L/D-D/L-Cᵢᵢ-X-X-X-X-S/H-X-Cᵢᵢᵢ;
(SEQ ID NO: 25)
Cᵢ-P-H-A/L-G-R-Cᵢᵢ-D-G-P-P/L-T/V-Cᵢᵢᵢ;
and
(SEQ ID NO: 26)
Cᵢ-D/H-H/V-X-R-M-D-Cᵢᵢ-P/F-X-X-Cᵢᵢᵢ;
wherein X represents any amino acid and X₁ represents any amino acid or is absent and Cᵢ, Cᵢᵢ and Cᵢᵢᵢ, represent first, second and third cysteine residues, respectively or a pharmaceutically acceptable salt thereof.

10. The peptide ligand as defined in claim 9, wherein the peptide ligand of Cᵢ-X-H-X-X-R-T/L-D-Cᵢᵢ-X-X-X₁-Cᵢᵢᵢ (SEQ ID NO: 23) comprises an amino acid sequence selected from any one of SEQ ID NOS: 1, 3, 5-6, 8, 10-11, 17 and 20-21:
(SEQ ID NO: 1)
CᵢKHYGRTDCᵢᵢHDTCᵢᵢᵢ;
(SEQ ID NO: 3)
CᵢPHIGRTDCᵢᵢPPCᵢᵢᵢ;
(SEQ ID NO: 5)
CᵢRHSDRLDCᵢᵢLPCᵢᵢᵢ;
(SEQ ID NO: 6)
CᵢPHSLRLDCᵢᵢHDCᵢᵢᵢ;
(SEQ ID NO: 8)
CᵢRHTHRLDCᵢᵢTESCᵢᵢᵢ;
(SEQ ID NO: 10)
CᵢGHVGRLDCᵢᵢHIPCᵢᵢᵢ;
(SEQ ID NO: 11)
CᵢPHVHRLDCᵢᵢHAPCᵢᵢᵢ;
(SEQ ID NO: 17)
CᵢKHSGRTDCᵢᵢHDTCᵢᵢᵢ;
(SEQ ID NO: 20)
CᵢKHAGRTDCᵢᵢPPCᵢᵢᵢ;
and
(SEQ ID NO: 21)
CᵢRHAGRTDCᵢᵢPPCᵢᵢᵢ;
wherein Cᵢ, Cᵢᵢ and Cᵢᵢᵢ represent first, second and third cysteine residues, respectively, or a pharmaceutically acceptable salt thereof.

11. The peptide ligand as defined in claim 9, wherein the peptide ligand of Cᵢ-D/E-A/L-S/R-R/H-L/D-D/L-Cᵢᵢ-X-X-X-X-S/H-X-Cᵢᵢᵢ (SEQ ID NO: 24) comprises an amino acid sequence selected from any one of SEQ ID NOS: 2, 7 and 18-19:
(SEQ ID NO: 2)
CᵢDASRLDCᵢᵢVPSSSGCᵢᵢᵢ;
(SEQ ID NO: 7)
CᵢELRHDLCᵢᵢRSHDHWCᵢᵢᵢ;
(SEQ ID NO: 18)
CᵢDASRLDCᵢᵢPYSVSLCᵢᵢᵢ;
and
(SEQ ID NO: 19)
CᵢDASRLDCᵢᵢPVVSHLCᵢᵢᵢ;
wherein Cᵢ, Cᵢᵢ and Cᵢᵢᵢ represent first, second and third cysteine residues, respectively, or a pharmaceutically acceptable salt thereof.

12. The peptide ligand as defined in claim 9, wherein the peptide ligand of Cᵢ-P-H-A/L-G-R-Cᵢᵢ-D-G-P-P/L-T/V-Cᵢᵢᵢ; (SEQ ID NO: 25) comprises an amino acid sequence selected from any one of SEQ ID NOS: 9 and 22:
(SEQ ID NO: 9)
CᵢPHAGRCᵢᵢDGPPTCᵢᵢᵢ;
and
(SEQ ID NO: 22)
CᵢPHLGRCᵢᵢDGPLVCᵢᵢᵢ;
wherein Cᵢ, Cᵢᵢ and Cᵢᵢᵢ represent first, second and third cysteine residues, respectively, or a pharmaceutically acceptable salt thereof.

13. The peptide ligand as defined in claim 9, wherein the peptide ligand of Cᵢ-D/H-H/V-X-R-M-D-Cᵢᵢ-P/F-X-X-Cᵢᵢᵢ (SEQ ID NO: 26) comprises an amino acid sequence selected from any one of SEQ ID NOS: 12-16:
(SEQ ID NO: 12)
CᵢDHRRMDCᵢᵢPEVCᵢᵢᵢ;
(SEQ ID NO: 13)
CᵢDHRRMDCᵢᵢPTLCᵢᵢᵢ;
(SEQ ID NO: 14)
CᵢDHRRMDCᵢᵢPTNCᵢᵢᵢ;
(SEQ ID NO: 15)
CᵢDHTRMDCᵢᵢPHNCᵢᵢᵢ;
and
(SEQ ID NO: 16)
CᵢHVGRMDCᵢᵢFQECᵢᵢᵢ;
wherein Cᵢ, Cᵢᵢ and Cᵢᵢᵢ represent first, second and third cysteine residues, respectively, or a pharmaceutically acceptable salt thereof.

14. The peptide ligand as defined in claim 9, wherein the peptide ligand of Cᵢ-D/H-H/V-X-R-M-D-Cᵢᵢ-P/F-X-X-Cᵢᵢᵢ (SEQ ID NO: 26) comprises an amino acid sequence selected from
(SEQ ID NO: 27)
CᵢDHRRMDCᵢᵢPTACᵢᵢᵢ;
wherein Cᵢ, Cᵢᵢ and Cᵢᵢᵢ represent first, second and third cysteine residues, respectively, or a pharmaceutically acceptable salt thereof.

15. The peptide ligand as defined in claim 1 or claim 2, which comprises an amino acid sequence selected from:
(SEQ ID NO: 28)
CᵢHATRNMDCᵢᵢYTCᵢᵢᵢ;
(SEQ ID NO: 29)
CᵢPHLERLDCᵢᵢHDSCᵢᵢᵢ;
(SEQ ID NO: 30)
CᵢHKGRGDHCᵢᵢLTCᵢᵢᵢ;
(SEQ ID NO: 31)
CᵢSPLRMDCᵢᵢHTVSDTCᵢᵢᵢ;
(SEQ ID NO: 32)
CᵢHSFRTDCᵢᵢHNHCᵢᵢᵢ;
(SEQ ID NO: 33)
CᵢPESHYLCᵢᵢRLDHHHCᵢᵢᵢ;
(SEQ ID NO: 34)
CᵢHAYRTDCᵢᵢHDYCᵢᵢᵢ;
(SEQ ID NO: 35)
CᵢNPRSDAPCᵢᵢEPCᵢᵢᵢ;
(SEQ ID NO: 36)
CᵢRTDDYRDCᵢᵢDICᵢᵢᵢ;
(SEQ ID NO: 37)
CᵢPHI[dA]RTDCᵢᵢPPCᵢᵢᵢ;
(SEQ ID NO: 38)
CᵢPHIG[Agb]TDCᵢᵢPPCᵢᵢᵢ;
(SEQ ID NO: 39)
CᵢDAS[HArg]LDCᵢᵢVPSSS[dA]Cᵢᵢᵢ;
(SEQ ID NO: 40)
CᵢP[HArg]I[dA]RTDCᵢᵢPPCᵢᵢᵢ;
(SEQ ID NO: 41)
CᵢP[HArg]IG[HArg]TDCᵢᵢPPCᵢᵢᵢ;
(SEQ ID NO: 42)
CᵢPHIG[HArg]TDCᵢᵢPPCᵢᵢᵢ;
(SEQ ID NO: 43)
CiP[HArg]IGRTDCᵢᵢPPCᵢᵢᵢ;
(SEQ ID NO: 44)
CiGHV[dA]RLDCᵢᵢHIPCᵢᵢᵢ;
(SEQ ID NO: 45)
CᵢGHVG[HArg]LDCᵢᵢHIPCᵢᵢᵢ;
(SEQ ID NO: 46)
Cᵢ[dA]HVGRLDCᵢᵢHIPCᵢᵢᵢ;
(SEQ ID NO: 47)
CᵢG[HArg]V[dA]RLDCᵢᵢ[HArg]IPCᵢᵢᵢ;
(SEQ ID NO: 48)
CᵢGHVGRLDCᵢᵢ[HArg]IPCᵢᵢᵢ;
(SEQ ID NO: 49)
Cᵢ[dA]HV[dA]RLDCᵢᵢ[HArg]IPCᵢᵢᵢ;
(SEQ ID NO: 50)
CᵢGHV[dA]RLDCᵢᵢ[HArg]IPCᵢᵢᵢ;
(SEQ ID NO: 51)
CᵢGHVG[HArg]LDCᵢᵢ[HArg]IPCᵢᵢᵢ;
(SEQ ID NO: 52)
Cᵢ[dA]HVG[HArg]LDCᵢᵢHIPCᵢᵢᵢ;
(SEQ ID NO: 53)
CᵢDAS[HArg]LDCᵢᵢVPSSSGCᵢᵢᵢ;
and
(SEQ ID NO: 54)
CᵢHTRAHDCᵢᵢYWESIVCᵢᵢᵢ;
wherein Agb represents 2-amino-4-guanidinobutyric acid, HArg represents homoarginine, Cᵢ, Cᵢᵢ and Cᵢᵢᵢ represent first, second and third cysteine residues, respectively, or a pharmaceutically acceptable salt thereof.

16. The peptide ligand as defined in any one of claim 1, 2 or 9, which comprises an amino acid sequence selected from:
A-(SEQ ID NO: 1)-A (herein referred to as BCY2519);
A-(SEQ ID NO: 2)-A (herein referred to as BCY2521);
A-(SEQ ID NO: 3)-A (herein referred to as BCY2524);
A-(SEQ ID NO: 4) (herein referred to as BCY2527);
A-(SEQ ID NO: 5) (herein referred to as BCY2528);
A-(SEQ ID NO: 6)-A (herein referred to as BCY2533);
A-(SEQ ID NO: 7)-A (herein referred to as BCY2534);
A-(SEQ ID NO: 8)-A (herein referred to as BCY2540);
A-(SEQ ID NO: 9)-A (herein referred to as BCY2541);
A-(SEQ ID NO: 10)-A (herein referred to as BCY2550);
A-(SEQ ID NO: 11)-A (herein referred to as BCY2552);
A-(SEQ ID NO: 12)-A (herein referred to as BCY2544);
A-(SEQ ID NO: 13)-A (herein referred to as BCY2545);
A-(SEQ ID NO: 14)-A (herein referred to as BCY2546);
A-(SEQ ID NO: 15)-A (herein referred to as BCY2547);
A-(SEQ ID NO: 16)-A (herein referred to as BCY2548);
A-(SEQ ID NO: 17)-A (herein referred to as BCY2520);
A-(SEQ ID NO: 18)-A (herein referred to as BCY2522);
A-(SEQ ID NO: 19)-A (herein referred to as BCY2523);
A-(SEQ ID NO: 20)-A (herein referred to as BCY2525);
A-(SEQ ID NO: 21)-A (herein referred to as BCY2526); and
A-(SEQ ID NO: 22)-A (herein referred to as BCY2542).

17. The peptide ligand as defined in any one of claim 1, 2 or 9, which comprises an amino acid sequence selected from:
A-(SEQ ID NO: 27)-A (herein referred to as BCY2543).

18. The peptide ligand as defined in claim 16, wherein the molecular scaffold is selected from 1,1′,1″-(1,3,5-triazinane-1,3,5-triyl)triprop-2-en-1-one (TATA) and the peptide ligand comprises an amino acid sequence selected from:
A-(SEQ ID NO: 1)-A (herein referred to as BCY2519);
A-(SEQ ID NO: 2)-A (herein referred to as BCY2521);
A-(SEQ ID NO: 3)-A (herein referred to as BCY2524);
A-(SEQ ID NO: 4) (herein referred to as BCY2527);
A-(SEQ ID NO: 5) (herein referred to as BCY2528);
A-(SEQ ID NO: 6)-A (herein referred to as BCY2533);
A-(SEQ ID NO: 7)-A (herein referred to as BCY2534);
A-(SEQ ID NO: 8)-A (herein referred to as BCY2540);
A-(SEQ ID NO: 9)-A (herein referred to as BCY2541);
A-(SEQ ID NO: 10)-A (herein referred to as BCY2550);
A-(SEQ ID NO: 11)-A (herein referred to as BCY2552);
A-(SEQ ID NO: 12)-A (herein referred to as BCY2544);
A-(SEQ ID NO: 13)-A (herein referred to as BCY2545);
A-(SEQ ID NO: 14)-A (herein referred to as BCY2546);
A-(SEQ ID NO: 15)-A (herein referred to as BCY2547);
A-(SEQ ID NO: 16)-A (herein referred to as BCY2548);
A-(SEQ ID NO: 17)-A (herein referred to as BCY2520);
A-(SEQ ID NO: 18)-A (herein referred to as BCY2522);
A-(SEQ ID NO: 19)-A (herein referred to as BCY2523);
A-(SEQ ID NO: 20)-A (herein referred to as BCY2525);
A-(SEQ ID NO: 21)-A (herein referred to as BCY2526); and
A-(SEQ ID NO: 22)-A (herein referred to as BCY2542).

19. The peptide ligand as defined in claim 17, wherein the molecular scaffold is selected from 1,1′,1″-(1,3,5-triazinane-1,3,5-triyl)triprop-2-en-1-one (TATA) and the peptide ligand comprises an amino acid sequence selected from:
A-(SEQ ID NO: 27)-A (herein referred to as BCY2543).

20. The peptide ligand as defined in claim 1 or claim 2, which additionally comprises a fluorescent moiety such as fluorescein (Fl) or Cyanine5 (Cy5) and is selected from:
A-(SEQ ID NO: 1)-A-Sar₆-K-Fl (herein referred to as BCY2594);
A-(SEQ ID NO: 2)-A-Sar₆-K-Fl (herein referred to as BCY2595);
Ac-(SEQ ID NO: 2)-A-Sar₆-K-Cy5 (herein referred to as BCY8589);
A-(SEQ ID NO: 3)-A-Sar₆-K-Fl (herein referred to as BCY2596);
Ac-(SEQ ID NO: 3)-A-Sar₆-K-Fl (herein referred to as BCY7508);
(SEQ ID NO: 4)-A-Sar₆-K-Fl (herein referred to as BCY2597);
A-(SEQ ID NO: 5)-A-Sar₆-K-Fl (herein referred to as BCY2598);
A-(SEQ ID NO: 6)-A-Sar₆-K-Fl (herein referred to as BCY2603);
A-(SEQ ID NO: 7)-A-Sar₆-K-Fl (herein referred to as BCY2604);
A-(SEQ ID NO: 8)-A-Sar₆-K-Fl (herein referred to as BCY2610);
Ac-(SEQ ID NO: 10)-A-Sar₆-K-Fl (herein referred to as BCY7509);
A-(SEQ ID NO: 28)-A-Sar₆-K-Fl (herein referred to as BCY2599);
A-(SEQ ID NO: 29)-A-Sar₆-K-Fl (herein referred to as BCY2600);
A-(SEQ ID NO: 30)-A-Sar₆-K-Fl (herein referred to as BCY2601);
A-(SEQ ID NO: 31)-A-Sar₆-K-Fl (herein referred to as BCY2602);
A-(SEQ ID NO: 32)-A-Sar₆-K-Fl (herein referred to as BCY2605);
A-(SEQ ID NO: 33)-A-Sar₆-K-Fl (herein referred to as BCY2606);
A-(SEQ ID NO: 34)-A-Sar₆-K-Fl (herein referred to as BCY2607);
A-(SEQ ID NO: 35)-A-Sar₆-K-Fl (herein referred to as BCY2608);
A-(SEQ ID NO: 36)-A-Sar₆-K-Fl (herein referred to as BCY2609);
Ac-(SEQ ID NO: 37)-A-Sar₆-K-Fl (herein referred to as BCY7503);
Ac-(SEQ ID NO: 37)-A-Sar₆-K-Cy5 (herein referred to as BCY8593);
Ac-(SEQ ID NO: 38)-A-Sar₆-K-Fl (herein referred to as BCY7502);
Ac-(SEQ ID NO: 39)-A-Sar₆-K-Fl (herein referred to as BCY7501);
Ac-(SEQ ID NO: 40)-A-Sar₆-K-Fl (herein referred to as BCY7504);
Ac-(SEQ ID NO: 41)-A-Sar₆-K-Fl (herein referred to as BCY7505);
Ac-(SEQ ID NO: 42)-A-Sar₆-K-Fl (herein referred to as BCY7506);
Ac-(SEQ ID NO: 43)-A-Sar₆-K-Fl (herein referred to as BCY7507);
Ac-(SEQ ID NO: 44)-A-Sar₆-K-Fl (herein referred to as BCY7510);
Ac-(SEQ ID NO: 44)-A-Sar₆-K-Cy5 (herein referred to as BCY8592);
Ac-(SEQ ID NO: 45)-A-Sar₆-K-Fl (herein referred to as BCY7511);
Ac-(SEQ ID NO: 46)-A-Sar₆-K-Fl (herein referred to as BCY7512);
Ac-(SEQ ID NO: 47)-A-Sar₆-K-Fl (herein referred to as BCY7513);
Ac-(SEQ ID NO: 48)-A-Sar₆-K-Fl (herein referred to as BCY7514);
Ac-(SEQ ID NO: 49)-A-Sar₆-K-Fl (herein referred to as BCY7515);
Ac-(SEQ ID NO: 50)-A-Sar₆-K-Fl (herein referred to as BCY7516);
Ac-(SEQ ID NO: 51)-A-Sar₆-K-Fl (herein referred to as BCY7517);
Ac-(SEQ ID NO: 52)-A-Sar₆-K-Fl (herein referred to as BCY7518);
Ac-(SEQ ID NO: 53)-A-Sar₆-K-Fl (herein referred to as BCY7764); and
Ac-(SEQ ID NO: 54)-A-Sar₆-K-Cy5 (herein referred to as BCY8588).

21. The peptide ligand as defined in any one of claims 1 to 20, wherein the pharmaceutically acceptable salt is selected from the free acid or the sodium, potassium, calcium, ammonium salt.

22. The peptide ligand as defined in any one of claims 1 to 21, wherein the integrin αvβ3 is human integrin αvβ3.

23. A drug conjugate comprising a peptide ligand as defined in any one of claims 1 to 19 and 21 to 22, conjugated to one or more effector and/or functional groups.

24. The drug conjugate comprising a peptide ligand as defined in any one of claims 1 to 19 and 21 to 22, conjugated to one or more cytotoxic agents.

25. A pharmaceutical composition which comprises the peptide ligand of any one of claims 1 to 19 or 21 to 22 or the drug conjugate of claim 23 or claim 24, in combination with one or more pharmaceutically acceptable excipients.

26. The peptide ligand as defined in any one of claims 1 to 19 or 21 to 22 or the drug conjugate as defined in claim 23 or claim 24, for use in preventing, suppressing or treating a disease or disorder mediated by integrin αvβ3.


SEQUENCE LISTING (58 sequences)

SEQ ID NO: 1 - protein, 13 aa, Artificial
  Synthetic Peptide
CKHYGRTDCHDTC

SEQ ID NO: 2 - protein, 15 aa, Artificial
  Synthetic Peptide
CDASRLDCVPSSSGC

SEQ ID NO: 3 - protein, 12 aa, Artificial
  Synthetic Peptide
CPHIGRTDCPPC

SEQ ID NO: 4 - protein, 15 aa, Artificial
  Synthetic Peptide
CYDDCRRLDHWQHSC

SEQ ID NO: 5 - protein, 12 aa, Artificial
  Synthetic Peptide
CRHSDRLDCLPC

SEQ ID NO: 6 - protein, 12 aa, Artificial
  Synthetic Peptide
CPHSLRLDCHDC

SEQ ID NO: 7 - protein, 15 aa, Artificial
  Synthetic Peptide
CELRHDLCRSHDHWC

SEQ ID NO: 8 - protein, 13 aa, Artificial
  Synthetic Peptide
CRHTHRLDCTESC

SEQ ID NO: 9 - protein, 13 aa, Artificial
  Synthetic Peptide
CPHAGRCDGPPTC

SEQ ID NO: 10 - protein, 13 aa, Artificial
  Synthetic Peptide
CGHVGRLDCHIPC

SEQ ID NO: 11 - protein, 13 aa, Artificial
  Synthetic Peptide
CPHVHRLDCHAPC

SEQ ID NO: 12 - protein, 12 aa, Artificial
  Synthetic Peptide
CDHRRMDCPEVC

SEQ ID NO: 13 - protein, 12 aa, Artificial
  Synthetic Peptide
CDHRRMDCPTLC

SEQ ID NO: 14 - protein, 12 aa, Artificial
  Synthetic Peptide
CDHRRMDCPTNC

SEQ ID NO: 15 - protein, 12 aa, Artificial
  Synthetic Peptide
CDHTRMDCPHNC

SEQ ID NO: 16 - protein, 12 aa, Artificial
  Synthetic Peptide
CHVGRMDCFQEC

SEQ ID NO: 17 - protein, 13 aa, Artificial
  Synthetic Peptide
CKHSGRTDCHDTC

SEQ ID NO: 18 - protein, 15 aa, Artificial
  Synthetic Peptide
CDASRLDCPYSVSLC

SEQ ID NO: 19 - protein, 15 aa, Artificial
  Synthetic Peptide
CDASRLDCPVVSHLC

SEQ ID NO: 20 - protein, 12 aa, Artificial
  Synthetic Peptide
CKHAGRTDCPPC

SEQ ID NO: 21 - protein, 12 aa, Artificial
  Synthetic Peptide
CRHAGRTDCPPC

SEQ ID NO: 22 - protein, 13 aa, Artificial
  Synthetic Peptide
CPHLGRCDGPLVC

SEQ ID NO: 23 - protein, 13 aa, Artificial
  Synthetic Peptide
  Xaa (2)..(2): Xaa represents any amino acid
  Xaa (4)..(5): Xaa represents any amino acid
  Xaa (7)..(7): Xaa is T or L
  Xaa (10)..(11): Xaa represents any amino acid
  Xaa (12)..(12): Xaa represents any amino acid or is absent
CXHXXRXDCXXXC

SEQ ID NO: 24 - protein, 15 aa, Artificial
  Synthetic Peptide
  Xaa (2)..(2): Xaa is D or E
  Xaa (3)..(3): Xaa is A or L
  Xaa (4)..(4): Xaa is S or R
  Xaa (5)..(5): Xaa is R or H
  Xaa (6)..(6): Xaa is L or D
  Xaa (7)..(7): Xaa is D or L
  Xaa (9)..(12): Xaa represents any amino acid
  Xaa (13)..(13): Xaa is S or H
  Xaa (14)..(14): Xaa represents any amino acid
CXXXXXXCXXXXXXC

SEQ ID NO: 25 - protein, 13 aa, Artificial
  Synthetic Peptide
  Xaa (4)..(4): Xaa is A or L
  Xaa (11)..(11): Xaa is P or L
  Xaa (12)..(12): Xaa is T or V
CPHXGRCDGPXXC

SEQ ID NO: 26 - protein, 12 aa, Artificial
  Synthetic Peptide
  Xaa (2)..(2): Xaa is D or H
  Xaa (3)..(3): Xaa is H or V
  Xaa (4)..(4): Xaa represents any amino acid
  Xaa (9)..(9): Xaa is P or F
  Xaa (10)..(11): Xaa represents any amino acid
CXXXRMDCXXXC

SEQ ID NO: 27 - protein, 12 aa, Artificial
  Synthetic Peptide
CDHRRMDCPTAC

SEQ ID NO: 28 - protein, 12 aa, Artificial
  Synthetic Peptide
CHATRNMDCYTC

SEQ ID NO: 29 - protein, 13 aa, Artificial
  Synthetic Peptide
CPHLERLDCHDSC

SEQ ID NO: 30 - protein, 12 aa, Artificial
  Synthetic Peptide
CHKGRGDHCLTC

SEQ ID NO: 31 - protein, 15 aa, Artificial
  Synthetic Peptide
CSPLRMDCHTVSDTC

SEQ ID NO: 32 - protein, 12 aa, Artificial
  Synthetic Peptide
CHSFRTDCHNHC

SEQ ID NO: 33 - protein, 15 aa, Artificial
  Synthetic Peptide
CPESHYLCRLDHHHC

SEQ ID NO: 34 - protein, 12 aa, Artificial
  Synthetic Peptide
CHAYRTDCHDYC

SEQ ID NO: 35 - protein, 12 aa, Artificial
  Synthetic Peptide
CNPRSDAPCEPC

SEQ ID NO: 36 - protein, 12 aa, Artificial
  Synthetic Peptide
CRTDDYRDCDIC

SEQ ID NO: 37 - protein, 12 aa, Artificial
  Synthetic Peptide
CPHIARTDCPPC

SEQ ID NO: 38 - protein, 12 aa, Artificial
  Synthetic Peptide
  Xaa (6)..(6): Xaa is Agb
CPHIGXTDCPPC

SEQ ID NO: 39 - protein, 15 aa, Artificial
  Synthetic Peptide
  Xaa (5)..(5): Xaa is HArg
CDASXLDCVPSSSAC

SEQ ID NO: 40 - protein, 12 aa, Artificial
  Synthetic Peptide
  Xaa (3)..(3): Xaa is HArg
CPXIARTDCPPC

SEQ ID NO: 41 - protein, 12 aa, Artificial
  Synthetic Peptide
  Xaa (3)..(3): Xaa is HArg
  Xaa (6)..(6): Xaa is HArg
CPXIGXTDCPPC

SEQ ID NO: 42 - protein, 12 aa, Artificial
  Synthetic Peptide
  Xaa (6)..(6): Xaa is HArg
CPHIGXTDCPPC

SEQ ID NO: 43 - protein, 12 aa, Artificial
  Synthetic Peptide
  Xaa (3)..(3): Xaa is HArg
CPXIGRTDCPPC

SEQ ID NO: 44 - protein, 13 aa, Artificial
  Synthetic Peptide
CGHVARLDCHIPC

SEQ ID NO: 45 - protein, 13 aa, Artificial
  Synthetic Peptide
  Xaa (6)..(6): Xaa is HArg
CGHVGXLDCHIPC

SEQ ID NO: 46 - protein, 13 aa, Artificial
  Synthetic Peptide
CAHVGRLDCHIPC

SEQ ID NO: 47 - protein, 13 aa, Artificial
  Synthetic Peptide
  Xaa (3)..(3): Xaa is HArg
  Xaa (10)..(10): Xaa is HArg
CGXVARLDCXIPC

SEQ ID NO: 48 - protein, 13 aa, Artificial
  Synthetic Peptide
  Xaa (10)..(10): Xaa is HArg
CGHVGRLDCXIPC

SEQ ID NO: 49 - protein, 13 aa, Artificial
  Synthetic Peptide
  Xaa (10)..(10): Xaa is HArg
CAHVARLDCXIPC

SEQ ID NO: 50 - protein, 13 aa, Artificial
  Synthetic Peptide
  Xaa (10)..(10): Xaa is HArg
CGHVARLDCXIPC

SEQ ID NO: 51 - protein, 13 aa, Artificial
  Synthetic Peptide
  Xaa (6)..(6): Xaa is HArg
  Xaa (10)..(10): Xaa is HArg
CGHVGXLDCXIPC

SEQ ID NO: 52 - protein, 13 aa, Artificial
  Synthetic Peptide
  Xaa (6)..(6): Xaa is HArg
CAHVGXLDCHIPC

SEQ ID NO: 53 - protein, 15 aa, Artificial
  Synthetic Peptide
  Xaa (5)..(5): Xaa is HArg
CDASXLDCVPSSSGC

SEQ ID NO: 54 - protein, 15 aa, Artificial
  Synthetic Peptide
CHTRAHDCYWESIVC

SEQ ID NO: 55 - protein, 6 aa, Artificial
  Synthetic Peptide
GRGDSP

SEQ ID NO: 56 - protein, 15 aa, Artificial
  Synthetic Peptide
ACLDHMECRGDMDCA

SEQ ID NO: 57 - protein, 15 aa, Artificial
  Synthetic Peptide
ACILRPNCDLDGRCA

SEQ ID NO: 58 - protein, 6 aa, Artificial
  Synthetic Peptide
GRGSSP